(19) United States
(12) Patent Application Publication
(10) Pub. No.: US 2020/0093408 A1
(43) Pub. Date: Mar. 26, 2020

(54) SYSTEMS, METHODS, AND SENSOR DEVICES FOR MEASURING CHANGES IN ANALYTE-SENSITIVE HYDROGELS

(71) Applicants: Florian Solzbacker, Salt Lake City, UT (US); Stan Kanarowski, Salt Lake City, UT (US); Jules John Magda, Salt Lake City, UT (US); Mahender Nath Avula, Salt Lake City, UT (US); Tatjana S. Bevans, Salt Lake City (US); Nassir F. Marrouche, Salt Lake City, UT (US); Derek J. Sakata, Salt Lake City, UT (US); Julia Koerner, Salt Lake City, UT (US); Christopher Reiche, Salt Lake City, UT (US)

(72) Inventors: Florian Solzbacker, Salt Lake City, UT (US); Stan Kanarowski, Salt Lake City, UT (US); Jules John Magda, Salt Lake City, UT (US); Mahender Nath Avula, Salt Lake City, UT (US); Tatjana S. Bevans, Salt Lake City (US); Nassir F. Marrouche, Salt Lake City, UT (US); Derek J. Sakata, Salt Lake City, UT (US); Julia Koerner, Salt Lake City, UT (US); Christopher Reiche, Salt Lake City, UT (US)

(21) Appl. No.: 16/621,510

(22) PCT Filed: Jun. 12, 2018
(86) PCT No.: PCT/US2018/037166

§ 371 Date: Dec. 11, 2019

Related U.S. Application Data
(60) Provisional application No. 62/518,491, filed on Jun. 12, 2017.
(60) Provisional application No. 62/571,096, filed on Oct. 11, 2017.
(60) Provisional application No. 62/592,120, filed on Nov. 29, 2017.
(60) Provisional application No. 62/592,131, filed on Nov. 29, 2017.

(51) Int. Cl. A61B 5/145; G01N 27/12; G01N 27/74; A61B 5/053; H01F 5/00

(52) U.S. Cl. CPC A61B 5/14546; G01N 27/126; G01N 27/745; A61B 2562/028; H01F 5/00; A61B 2562/12; A61B 5/0538

(57) ABSTRACT

Systems, methods, and sensor devices for identifying one or more changes in a stimulus-responsive hydrogel include a sensor device having (i) a sensing structure and (ii) a stimulus-responsive hydrogel associated with a first side of the sensing structure. The sensing structure includes a flexible thin film polymer and an electric sensing element capable of electric impedance change, and the hydrogel is configured to dimensionally change in response to predefined stimuli such that a dimensional change of the hydrogel causes a change in an impedance property of the electric sensing element. Systems including such a sensor device can additionally include a meter in electrical communication with the sensor device to identify changes in the impedance properties of the structure and/or a catheter sheath configured for placement within an in vivo environment and is sized and shaped to receive the sensor device within a lumen thereof.

### CROSS-REFERENCE TO RELATED APPLICATIONS

This application claims priority to and the benefit of U.S. Provisional Patent Application No. 62/518,491, filed Jun. 12, 2017 and titled “METHODS TO DETECT VOLUME CHANGES OF HYDROGELS USING ULTRASOUND,” U.S. Provisional Patent Application Ser. No. 62/571,096, filed Oct. 11, 2017 and titled “HYDROGEL-BASED BIOMARKER SENSORS FOR USE DURING THE ADMINISTRATION OF ANESTHESIA, SEDATION OR FOR MEASURING OPIATE COMPLIANCE,” U.S. Provisional Patent Application Ser. No. 62/592,120, filed Nov. 29, 2017 and titled “DETECTION OF HYDROGEL VOLUME CHANGES BY MICROCOIL AND HYDROGEL-BASED BIOMARKER SENSORS FOR USE DURING THE ADMINISTRATION OF ANESTHESIA, SEDATION OR FOR MEASURING OPIATE COMPLIANCE,” and U.S. Provisional Patent Application No. 62/592,131, filed Nov. 29, 2017 and titled “DEVICE ARCHITECTURE PI SENSOR CROSS SECTION PROCESS FLOW.” All of the aforementioned are incorporated herein by reference in their entirety.

### BACKGROUND

### Technical Field

This disclosure generally relates to biomedical sensor devices. More specifically, the present disclosure relates to sensors that transduce volume changes of stimuli-responsive hydrogels into electric signals to create a biomedical sensor device.

### Related Technology

Advances in computing technology have resulted in concomitant advances in medical device technologies, including within the field of diagnostic and interventional medicine. Particularly, the past century has demonstrated significant advances in medical imaging devices that can be used, for example, to noninvasively view and explore internal structures of the body and to provide image guidance for any of a plethora of medical devices during surgical procedures.

While medical imaging technologies have allowed increased clarity and precision during surgical procedures and elsewhere, these technologies still lack the resolving power to identify the presence or absence—let alone the concentration—of biomarkers within the body. In most instances, healthcare providers still rely on manual, resource intensive assays to determine the presence, absence, or concentration of biomarkers within the body, and some of the assays or measurements obtained by current methods are indirect measures of a physiologic state. At best, these assays provide a glimpse into a past physiologic state and epitomize the lack of viable sensors for real-time monitoring of the concentration of biomarkers or medications in vivo.

Furthermore, patient compliance with prescribed medications can dramatically impact patient outcome during and following a medical procedure or life event. For example, a lack of patient compliance with a prescribed regimen can result in a failure to reach therapeutically relevant concentrations in the body, or to overdosing and abuse. With respect to the latter, opioid use and abuse is an epidemic in the United States and elsewhere. Opioid prescriptions increased from the mid-1990s through the mid-2010s with a coincident increase in opioid addiction and abuse. An estimated 1.9 million Americans have a substance use disorder involving painkillers, with a strong preference for opioid painkillers. As with many other medications and biomarkers, there are no available sensors that enable the real-time measurement or monitoring of opioid levels in a patient.

Greater clarity into the presence and concentration of medications and various biomarkers within a patient before, during, and after a procedure or life event could dramatically influence patient care and patient compliance. Accordingly, there are a number of disadvantages with biomedical sensor devices that can be addressed.

### BRIEF SUMMARY

Embodiments of the present disclosure solve one or more of the foregoing or other problems in the art of biomedical sensor devices. For example, systems, methods, and sensor devices for identifying one or more dimensional changes in a stimulus-responsive hydrogel positioned within an in vivo environment are disclosed and can be implemented, for example, to determine a concentration of a predefined analyte within the in vivo environment. Manufacturing methods for sensor devices and their use within various in vivo and in situ methods and systems for identifying and/or monitoring the presence and/or concentration of an analyte are additionally disclosed.

For example, embodiments of the present disclosure include sensor devices having (i) a sensing structure made up of at least a flexible thin film polymer and an electric sensing element capable of electric impedance change and (ii) a stimulus-responsive hydrogel associated with a first side of the sensing structure that is configured to dimensionally change in response to one or more predefined stimuli such that a dimensional change of the stimulus-responsive hydrogel causes a change in one or more impedance properties of the electric sensing element. In one embodiment, the electric sensing element is an inductor.

In one embodiment, the electric sensing element within a sensor device includes a conductive meandering lead embedded within the flexible thin film polymer substrate. The conductive meandering lead can include a metal thin film with the metal being selected from the group consisting of gold, platinum, titanium, aluminum, and alloys thereof In one embodiment, the variable electric impedance is realized as a three-dimensional stack of interconnected conductive meandering leads embedded within the flexible thin film polymer.

In one embodiment, the stimulus-responsive hydrogel of a sensor device is deposited on a distal end of the sensing structure such that the dimensional change of the stimulus-responsive hydrogel causes the change in the one or more impedance properties of the electric sensing element by bending the sensor substrate. Such sensor devices and other similar sensor devices disclosed herein can be included within a system for measuring one or more changes in an associated stimulus-responsive hydrogel that is positioned within an in vivo environment. In addition to the sensor device, the system can include a catheter sheath configured for placement within the in vivo environment and which is sized and shaped to receive the sensor device within the catheter sheath lumen. In some instances, the sensor device obstructs less than about 10% of the catheter sheath opening, preferably less than 5% of the catheter sheath opening, or more preferably less than about 2.5% of the catheter sheath opening.

Such sensor devices and other similar sensor devices disclosed herein can be manufactured using a method that includes the method acts of (i) depositing a first polymer layer of the flexible thin film polymer on a carrier wafer, (ii) selectively depositing a metal thin film pattern comprising the electric sensing element on a portion of the first polymer layer, (iii) depositing a second polymer layer of the flexible thin film polymer over the first polymer layer and the metal thin film pattern, (iv) fabricating one or more contact pads and creating an outline of the sensing structure such that the sensing structure additionally includes the one or more contact pads, (v) removing the sensing structure from the carrier wafer, (vi) chemically treating the first side of the sensing structure, and (vii) molding and conditioning the stimulus-responsive hydrogel on the first side of the sensing structure. In some aspects, the acts of selectively depositing the metal thin film pattern and fabricating the one or more contact pads and creating the outline of the sensing structure are performed using one or more lift-off processes.

In one embodiment, the stimulus-responsive hydrogel is deposited on the distal end of the sensing structure as one or more micro-strips, each of the one or more micro-strips being between about 2 μm-200 μm thick, about 25 μm-500 μm wide, and about 500 μm-1000 μm long. The flexible thin film polymer substrate can be fabricated of polyimide with a thickness between about 10 μm-20 μm.

Embodiments of the present disclosure additionally include systems for measuring one or more changes in a stimulus-responsive hydrogel positioned within an in vivo environment. The system can include (i) a sensing structure having an electric sensing element, (ii) a stimulus-responsive hydrogel associated with the sensing structure that is configured to dimensionally change in response to one or more predefined stimuli, and (iii) a meter in electrical communication with the sensing structure that is configured to identify a change in one or more impedance properties of the electric sensing element.

In one embodiment, the electric sensing element within the system includes a micro-coil, which can be connected to a capacitor as part of a resonance circuit within the sensing structure. In one aspect of the system, the micro-coil includes a soft metal wire having a diameter between about 5 μm_-50 μm. For example, the micro-coil can include a gold metal wire having a diameter of about 25 μm.

In one embodiment, the stimulus-responsive hydrogel of the system surrounds the micro-coil, and a dimensional change within the stimulus-responsive hydrogel causes a compression or lengthening of the micro-coil.

In one embodiment, the stimulus-responsive hydrogel of the system is a thin strand hydrogel core disposed within an internal space defined by the micro-coil, and a dimensional change within the stimulus-responsive hydrogel causes a change in magnetic permeability of the micro-coil. In one aspect, the thin strand hydrogel core includes a plurality of magnetic particles.

In one embodiment, the disclosed systems additionally include a computer system in electrical communication with the meter. The computer system includes at least one or more processors and is to receive, from the meter, a first impedance of the sensing structure at a first point in time and a second impedance of the sensing structure at a second point in time and to calculate, at the one or more processors, a volumetric change in the stimulus-responsive hydrogel based on the received first and second impedances.

Embodiments of the present disclosure additionally include methods for measuring a concentration of an analyte within an in vivo environment. In one embodiment, a method includes the acts of (i) positioning a sensor device within the in vivo environment where the sensor device includes a sensing structure and a stimulus-responsive hydrogel associated with the sensing structure and the stimulus-responsive hydrogel is configured to change one or more impedance properties of the sensing structure in response to the concentration of the analyte, (ii) receiving, from a meter in electrical communication with the sensor device, a first impedance of the sensing structure, (iii) measuring an impedance amplitude or phase change, preferably at one of the lead structure's resonance frequencies, and (iv) determining the concentration of the analyte based on the impedance amplitude or phase change.

In one embodiment, the method additionally includes the acts of (v) receiving, from the meter in electrical communication with the sensor device, a second impedance of the sensing structure, (vi) calculating an impedance change based on the first impedance and second impedance, and (vii) determining an updated concentration of the analyte based on the impedance change.

Accordingly, among other things, sensor devices for identifying one or more changes in a stimulus-responsive hydrogel, systems for measuring one or more changes in a stimulus-responsive hydrogel positioned within an in vivo environment, and methods for measuring a concentration of an analyte within an in vivo environment are disclosed.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

Additional features and advantages of the disclosure will be set forth in the description which follows, and in part will be obvious from the description, or may be learned by the practice of the disclosure. The features and advantages of the disclosure may be realized and obtained by means of the instruments and combinations particularly pointed out in the appended claims. These and other features of the present disclosure will become more fully apparent from the following description and appended claims or may be learned by the practice of the disclosure as set forth hereinafter.

### BRIEF DESCRIPTION OF THE DRAWINGS

In order to describe the manner in which the above recited and other advantages and features of the disclosure can be obtained, a more particular description of the disclosure briefly described above will be rendered by reference to specific embodiments thereof, which are illustrated in the appended drawings. It is appreciated that these drawings depict only typical embodiments of the disclosure and are not therefore to be considered to be limiting of its scope. The disclosure will be described and explained with additional specificity and detail through the use of the accompanying drawings in which:

FIG. 1 illustrates a diagram representing the analyte-concentration-dependent or stimulus-intensity-dependent swelling of hydrogels;

FIG. 2 illustrates a simplified model of molecular imprinting as relevant to generation of one or more types of stimulus-responsive hydrogels;

FIG. 3 illustrates a simplified model of the generation and reversible swelling of stimulus-responsive hydrogels that utilize antibody mediated biomarker recognition;

FIG. 4 illustrates a portion of a sensor device having a micro-coil associated with a stimulus-responsive hydrogel in accordance with embodiments of the present disclosure;

FIG. 5 illustrates a sensor device with a sensing structure having a flexible thin film polymer and an electric sensing element embedded therein and a hydrogel attached to one side of the sensing structure in accordance with embodiments of the present disclosure;

FIG. 6 illustrates a graph of the electrical impedance response measured as the reflective coefficient of an exemplary sensor device in accordance with embodiments of the present disclosure;

FIG. 7 illustrates an exemplary method for manufacturing sensor devices in accordance with embodiments of the present disclosure;

FIG. 8 illustrates a system for measuring changes in a stimulus-responsive hydrogel positioned within an in vivo environment that includes a sensor device and a standard catheter sheath to receive the sensor device within a lumen of the catheter sheath in accordance with embodiments of the present disclosure;

FIG. 9 illustrates a magnified view of the distal tip of the catheter sheath and associated sensor device of FIG. 8; and

FIG. 10 illustrates an exemplary application of disclosed sensor devices and accompanying systems for identifying one or more changes in a stimulus-responsive hydrogel positioned within an in vivo environment.

### DETAILED DESCRIPTION

Before describing various embodiments of the present disclosure in detail, it is to be understood that this disclosure is not limited to the parameters of the particularly exemplified systems, methods, apparatus, products, processes, and/or kits, which may, of course, vary. Thus, while certain embodiments of the present disclosure will be described in detail, with reference to specific configurations, parameters, components, elements, etc., the descriptions are illustrative and are not to be construed as limiting the scope of the claimed invention. In addition, the terminology used herein is for the purpose of describing the embodiments and is not necessarily intended to limit the scope of the claimed invention.

### Hydrogel Sensing

As discussed above, medical imaging technologies have allowed increased clarity and precision during surgical procedures and elsewhere, but these technologies lack the resolving power to identify the presence or concentration of analytes within the body. In most instances, healthcare providers still rely on manual, resource intensive assays to determine the presence, absence or concentration of biomarkers or other analytes within the body, and some of the assays or measurements obtained by current methods are indirect measures of a physiologic state. At best, these assays provide a glimpse into a past physiologic state and epitomize the lack of viable sensors for real-time monitoring of the concentration of biomarkers, medications, or other analytes in vivo. Other advancements, however, can be adapted for this purpose. One such example includes hydrogels.

Hydrogels are objects consisting of hydrophilic cross-linked networks of polymer that have both liquidlike and solidlike properties. Smart hydrogels characteristically experience a change in their volume and mechanical properties in response to the presence of a specific stimulus. As used herein, the term “stimulus” includes any force, environmental condition (e.g., temperature, pH, osmolarity, humidity, etc.), or analyte. An “analyte,” as used herein, includes any substance that can itself be identified or measured or of which a chemical or physical property thereof can be identified or measured. Analytes include, for example, nucleic acids, proteins, chemicals, or other compounds. In some instances, analytes serve as a physiologic, pathologic, or environmental marker of a known or unknown phenomenon (e.g., insulin levels can serve as a biomarker for diabetes). Analytes are additionally understood to include pharmaceutical compounds, their physiologic byproducts, or compounds derived therefrom. Hydrogels that are responsive to one or more stimuli are termed “stimulus-responsive hydrogels,” which encompass the subsets of “analyte-sensitive hydrogels” and “biomarker-sensitive hydrogels.” Although portions of the disclosure particularly recite an “analyte-sensitive hydrogel” or “biomarker-sensitive hydrogel,” it should be appreciated that the disclosed embodiments apply generally to smart hydrogels that are responsive to any stimulus unless specifically stated otherwise.

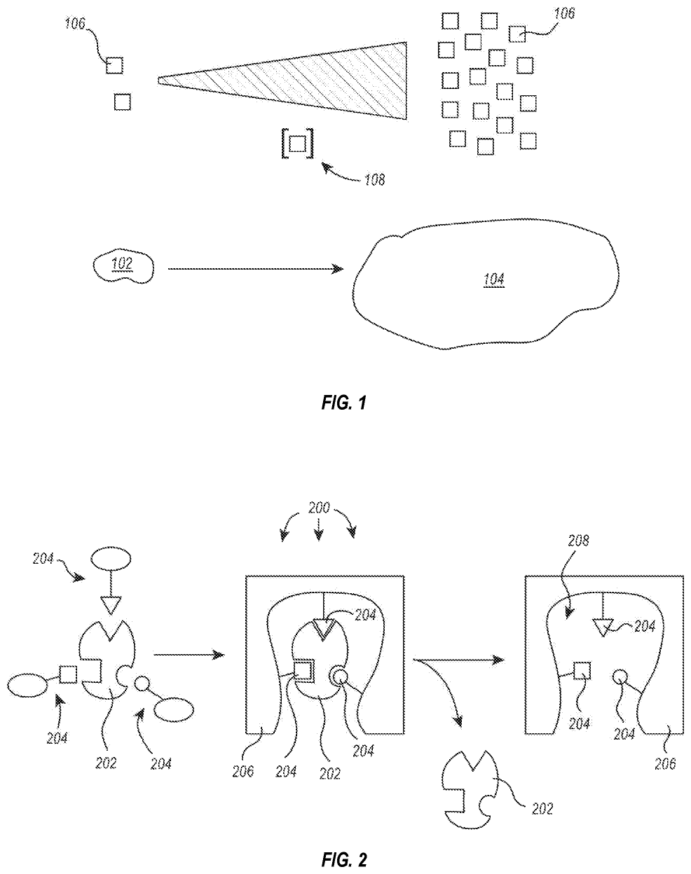

FIG. 1 illustrates the characteristic change in volume exhibited by stimulus-responsive hydrogels. As shown, a stimulus-responsive hydrogel can transition from a collapsed or shrunken state 102 to a swollen state 104 in response to the presence of a specific stimulus or analyte 106. Although seemingly depicted as a binary transition—from a shrunken state 102 to a swollen state 104—the change in volume exhibited by hydrogels is typically not binary, just as a change in analyte availability is also often not represented as a binary modality. As shown, the concentration 108 of the analyte 106 can span any point along a spectrum from low to high concentration. Advantageously, hydrogels respond to variable analyte concentrations in a similar graded fashion. Instead of being a binary readout of the presence or absence of an analyte or stimulus, smart hydrogels experience a change in volume that correlates with the concentration of analyte interacting with the hydrogel. Hydrogels can additionally reversibly bind analyte, allowing them to shrink and swelling response to potential ebbs and flows of analyte concentration.

Hydrogels can sense analytes and/or stimuli using a variety of recognition domains, and the binding specificity and fidelity can be tuned to various sensitives, often depending on the type of recognition domain utilized. FIG. 2 illustrates a generalized method 200 of molecular imprinting that can be used to manufacture hydrogels with specificity for a given analyte. Molecular imprinting relies on a combination of reversible binding and shape complementarity to specifically recognize target analytes. Referring to FIG. 2, the first step of the illustrated method 200 is to allow functional monomers 204 to interact with and self-assemble about a print molecule 202. The print molecule 202 includes the same desired shape as the target analyte such that when the functional monomers 204 self-assemble and are polymerized, the resulting molecular imprinted polymer 206 includes a cavity 208 that is complementary in size and shape to the print molecule. The print molecules can be removed from the molecular imprinted polymer 206, leaving the cavity 208 to function as a selective binding site for the specific analyte.

Hydrogels can be made to include a variety of additional or alternative analyte recognition domains, such as antibodies or nucleic-acid-based aptamers. Antibodies traditionally demonstrate a high level of specificity for targets and are good receptors for biomarkers. Advantageously, antibody binding of biomarkers is a reversible process, and when incorporated into a hydrogel, the resultant biomarker-sensitive hydrogel can dynamically respond to changes in biomarker concentration and thereby dynamically change its volume (e.g., shrinking or swelling) in response to increased or decreased concentrations of available biomarker. As shown in FIG. 3, antibodies 304 can be added to a pre-polymerized hydrogel solution 302, and following polymerization, the antibodies 304 are positioned within the resultant biomarker-sensitive hydrogel 306 so as to interact with biomarkers 310. In some instances, lithography or similar manufacturing methods can be used to first form a foundation of polymerized hydrogel—into which the antibodies are arranged—followed by polymerization of the remaining hydrogel.

As additionally illustrated in FIG. 3, the biomarker-sensitive hydrogel 306 is configured to swell (e.g., as shown by swollen biomarker-sensitive hydrogel 308) in response to interacting with additional biomarkers 310 and shrink (e.g., as shown in biomarker-sensitive hydrogel 306) in response to a lower concentration of available biomarkers 310. Accordingly, the biomarker-sensitive hydrogel 306 is configured to reach an equilibrium 312 between swollen and shrunken states based on the amount or concentration of biomarker 310 available to interact with the antibodies 304 within the biomarker-sensitive hydrogel 306. In doing so, the biomarker-sensitive hydrogel 306 acts as a biosensor for the amount or concentration of biomarker 310 in the vicinity of the biomarker-sensitive hydrogel 306, and by determining the volume of the biomarker-sensitive hydrogel 306, the amount or concentration of biomarker can be determined.

The same principle applies to many smart hydrogels. That is, stimulus-responsive hydrogels can be made to various sensitivity levels and can be used as a type of sensor that reads out the concentration of analyte or level of stimulus based upon its volume. If the volume of a characterized hydrogel can be determined accurately, the concentration, or at the very least a change in concentration, can be calculated therefrom.

The possibility to tailor hydrogel responses to different analytes or stimuli has made hydrogels an attractive material for applications like drug delivery, sensing, actuation, and implants. Hydrogels, particularly analyte-sensitive hydrogels, are additionally attractive as a material for biosensing and even potentially as an implant due to its inherent biocompatibility, failure to elicit an immune response, and dynamic ability to correlate analyte concentrations within an in vivo system.

However, there remains an impediment in obtaining a real-time, sensitive, and reliable measurement of the volume of stimulus-responsive hydrogels, particularly when the hydrogels are implanted in vivo. Noninvasive medical imaging techniques would be an ideal method to measure hydrogel volume in vivo, but hydrogels are nearly invisible to most medical imaging devices. Previous detection methods include sensing of the swelling pressure of hydrogels using a piezo resistive pressure sensor or by measuring the deformation of cantilever beams. The latter, while highly sensitive, utilizes large laser-based detection setups and is, therefore, often unsuited for in vivo applications where the hydrogel and transducer are sufficiently compact to be used within an in vivo environment. Pressure sensors can be miniaturized, but due to their rather low sensitivity, a prohibitively large amount of hydrogel would need to be incorporated into such a sensor. These foregoing issues make it difficult to accurately determine any response of hydrogels to surrounding analytes and/or stimuli using current technologies.

Thus, even though hydrogels represent a promising material for biomedical and biotechnological applications, their lack of visibility and concomitant lack of ability to be tracked in real time using current imaging devices and techniques renders their potential a moot point.

### Micro-Coil-Based Sensors for Identifying Changes of Stimulus-Responsive Hydrogels

Embodiments of the present disclosure solve one or more of the foregoing problems in the art of identifying changes in analyte concentration using stimulus-responsive hydrogels. For example, the swelling and shrinking properties of hydrogels can be used to alter the inductance (or other electrical property) of an electrical circuit, and that change in inductance can be used to determine the volumetric change in the hydrogel—and consequently the concentration of analyte affecting the hydrogel. Because the inductance of a micro-coil, one type of inductor, is proportional to the micro-coil's cross-sectional area, number of turns, and magnetic permeability and inversely proportional to the micro-coil's length, the swelling and contraction of an associated hydrogel can measurably alter the inductance properties of the micro-coil. The measured change in inductance can be extrapolated to a concomitant change in analyte concentration or stimulus intensity. The relatively small footprint of a micro-coil makes such embodiments beneficial for in vivo use.

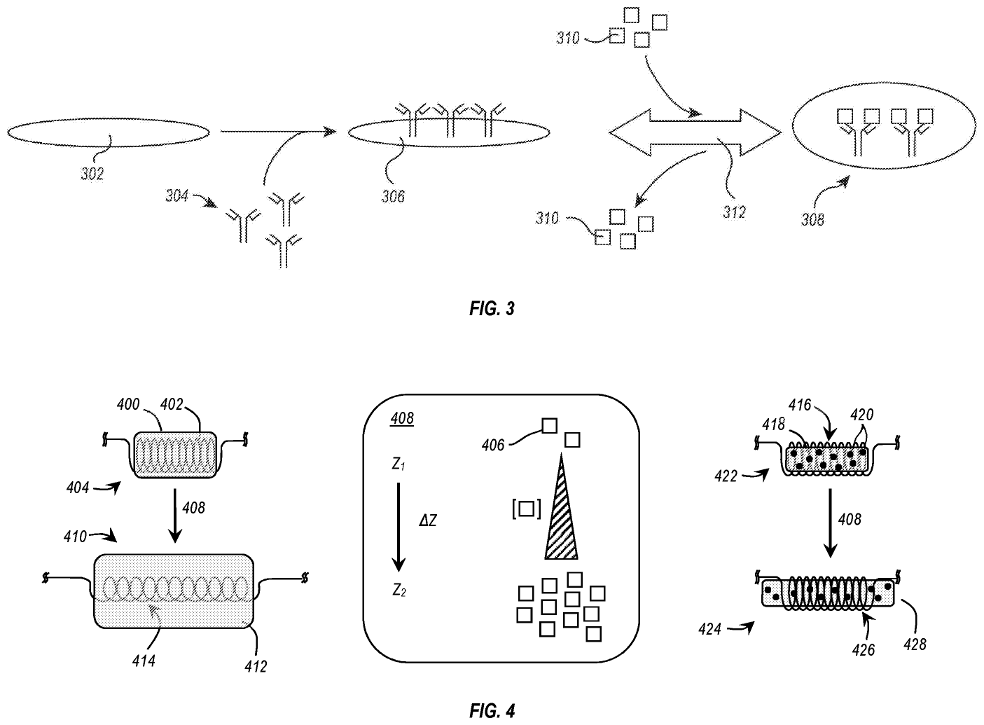

For example, as shown in FIG. 4, a stimulus-responsive hydrogel 400 polymerized around a micro-coil 402 has an initial volume 404 at a first concentration of analyte 406. At this initial volume 404, the micro-coil 402 has an initial inductance (Z1). Upon a change in analyte concentration 408, the stimulus-responsive hydrogel 400 transitions to a second volume 410, becoming a swollen stimulus-responsive hydrogel 412. The micro-coil 402 is similarly altered by the swollen stimulus-responsive hydrogel 412, causing one or more properties of the micro-coil 402 to change. The resulting altered micro-coil 414 can be lengthened (as shown) and/or undergo a change in its cross-sectional area (also shown). These changes in the micro-coil 414 result in a change in inductance (ΔZ) from the initial inductance (Z₁) to a second measurable inductance (Z₂), from which a volumetric change in the hydrogel can be calculated and a change in concentration of analyte inferred.

It should be appreciated that similar changes can occur in the reverse order. For example, a hydrogel can shrink in response to a decreased concentration of analyte and by so doing compress the micro-coil. The compressed micro-coil can have a shorter length and/or increased cross-sectional area compared to its previous, uncompressed state. Such changes in the properties of the micro-coil can cause observable changes in its inductance properties. Such a configuration is advantageous over other techniques as the stimulus-responsive hydrogel surrounds the micro-coil and therefore reduces and/or eliminates any obstruction that would prevent contact between the hydrogel and the solution containing the analytes to be measured.

In some embodiments, the micro-coil is constructed of a thin wire between about 5 μ-50 μm thick, preferably between about 10 μm-35 μm thick, or more preferably about 25 μm thick and made of a soft metal such as gold. The micro-coil can additionally be constructed with a pitch between the turns sot that the coil can easily be deformed by volumetric changes in the associated stimulus-responsive hydrogel.

The micro-coil 402, 414 of FIG. 4 can be connected to a capacitor to form a resonance circuit. An inductance change of the micro-coil will alter the resonance frequency of the circuit, which can be detected wireless using, for example, an RFID-like mechanism. Alternatively, the circuit containing the micro-coil 402, 414 can be electrically coupled to an inductance meter or other instrument for measuring electrical properties of a circuit, or portions of a circuit, as known in the art. The inductance meter can thereby directly measure changes in one or more properties of the micro-coil.

Additionally illustrated in FIG. 4 is a stimulus-responsive hydrogel 418 having a plurality of magnetic particles 420 (e.g., iron oxide particles) that forms a variable core within the micro-coil 416. At a first concentration of analyte 406, the stimulus-responsive hydrogel core 418 has a first volume 422, and the magnetic particles 420 slightly increase the magnetic permeability of the micro-coil 418, which in turn increases the inductance of the micro-coil 416. As the volume of the stimulus-responsive hydrogel core 418 changes in response to changing analyte concentration/stimulus intensity, the diameter and/or length of the core 418 is altered. A change in length can affect the inductance of the micro-coil 416, which will, in turn, affect the resonance frequency of a resonance circuit that includes the micro-coil 418 and an adjusted capacitor (not shown). Such changes in resonance frequency can be detected using the same or similar mechanisms and methods discussed above.

The foregoing embodiments provide certain advantages. For example, as shown in FIG. 4 the micro-coil can be manufactured to be mechanically stable enough to counteract the force of a radially expanding hydrogel while still allowing changes in the hydrogel (e.g., changes in length) to cause a measurable change in properties of the micro-coil, namely changes in the magnetic permeability of the micro-coil. It is additionally advantageous as the micro-coil can constrain the strand-like hydrogel core and prevent the buckling and curling observed for unconstrained strand-like hydrogels.

### Thin-Film-Based Sensors for Identifying Changes of Stimulus-Responsive Hydrogels

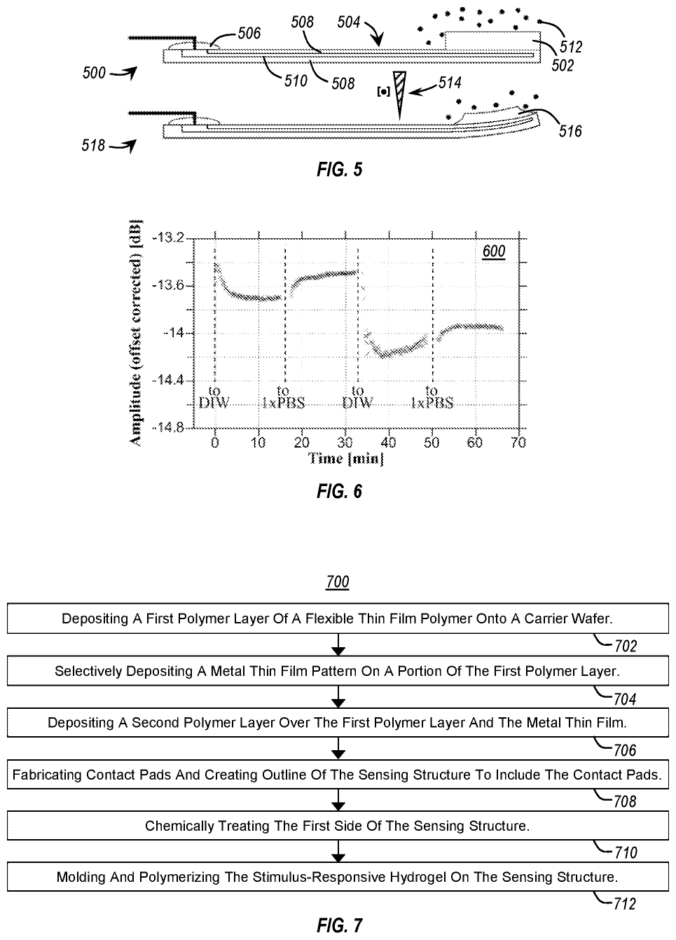

The foregoing micro-coil-based sensors solve one or more problems in the art of hydrogel sensing by offering a reliable, robust sensor for accurately and easily identifying the concentration of analyte, particularly within an in vivo environment. However, the dimensions of the micro-coil can limit its implementation when strong spatial restrictions exist. An exemplary low-profile sensor incorporating one or more disclosed embodiments is illustrated in FIG. 5 and additionally solves one or more problems in the art of hydrogel sensing, including use in situations with strong spatial restrictions. In general, the sensor device of FIG. 5 identifies changes in the impedance properties of a metal lead embedded within a flexible, thin film polymer as the polymer is stretched and/or bent in response to swelling of an associated thin, stimulus-responsive hydrogel. An analyte concentration (for which the hydrogel is sensitive) can then be inferred from the change in impedance.

As shown in FIG. 5, the sensor 500 includes a stimulus-responsive hydrogel 502 attached to a distal end of one side of a sensing structure 504. The sensing structure 504 includes a flexible and/or stretchable polymer film 508 that has an embedded metallic thin film structure 510. The embedded metallic thin film structure 508 includes a sensing portion proximate the associated hydrogel 502 (e.g., at or near the distal end of the sensor 500) and one or more contact pads 506 configured as coupling points between the sensor and an external electronic device such as a meter for measuring one or more electrical properties of the metallic thin film structure 508, particularly the sensing portion thereof.

In some embodiments, the sensing portion of the metallic thin film structure is made up of or includes conductive meandering leads that act as electric sensing elements. When the stimulus-responsive hydrogel 512 responds to a change in analyte (or stimulus intensity) 514, it causes a volume change in the hydrogel 516 which stretches and/or bends (e.g., in a bimetal like fashion) the flexible polymer film 508. This deformation of the flexible polymer film 508 causes a similar local deformation of the embedded metallic thin film structure 510, which results in an impedance change in the altered sensor device 518. For example, the impedance change can be detected as an amplitude and phase change of the measured sensor device output voltage at high frequencies (e.g., at or above 1 MHz, preferably above 10 MHz), which can be employed as the measurement signal. In some embodiments, the frequency used is at or close to one of the electrical resonance frequencies of the sensor 500, though it should be appreciated that other frequencies can be used as well. Furthermore, the change of one of the sensing structure's resonance frequencies can be measured.

FIG. 6 illustrates a graph 600 of an electrical impedance response of an exemplary sensor device having a stimulus-responsive hydrogel that is responsive to ionic strength. As shown, the reflection coefficient of the metallic lead structure was measured with a network analyzer at a frequency of 163.5 MHz. An offset that was induced each time the liquid was replaced was subtracted. As can be seen in FIG. 6, a measurable change in the reflective coefficient occurred when switching the sensor structure between deionized water (DIW) and phosphate buffered saline (PBS), indicating that the bending of the flexible polymer substrate and embedded conductive meandering leads occurred in response to the associated hydrogel contracting and expanding in solutions of different ionic strengths. The same or similar response can be observed for stimulus-responsive hydrogels having different stimulus or analyte specificities. Furthermore, although the data presented within FIG. 6 illustrates a binary sensing modality of the tested sensor device, it should be appreciated that such sensor device—and other sensor devices disclosed and envisioned herein—can have a concentration- or stimulus-intensity-dependent change in one or more electrical properties of the incorporated electric sensing element, including impedance changes.

When detected, a measured impedance change can be used to determine a commensurate volumetric change in the associated hydrogel—and thereby the concentration of analyte detected by the hydrogel. In some embodiments, a calibration measurement or simulation can be used to provide context or a framework from which the impedance change can be translated into a change in concentration of analyte observed by the stimulus-responsive hydrogel.

An important parameter of a desired sensor device, particularly one intended for use in an in vivo environment, is a fast response time—the amount of time it takes for the sensor device to equilibrate to an initial analyte concentration and/or the amount of time it takes for the associated hydrogel to detect a change in analyte concentration and for that detected change to be translated into a measurable electric signal. With respect to stimulus responsive hydrogels, the speed of the volume change is diffusion limited, and therefore, depending on the size of the hydrogel, it can take minutes or hours for a detectable volume change to occur in the hydrogel. In some embodiments, the hydrogel 502 associated with the sensor device 500 is about 20 μm thick and can take seconds to minutes to measurably respond to a change in analyte concentration. The thickness of the hydrogel can be selected based on sensitivity and/or desired response time and are typically between about 1 μm-400 μm thick. In some embodiments, the hydrogel is between 100 μm-200 μm thick and has a response time of less than about 10 minutes. In some embodiments, the thickness of a hydrogel is between about 25 μm-50 μm thick and has a response time of less than about five minutes. In some embodiments, the thickness of a hydrogel is between about 5 μm-25 μm and has a response time of less than two minutes. In some embodiments, the hydrogel is less than 10 μm thick and has a response time of one minute or less.

In some embodiments, the sensing structure is very thin—less than about 50 μm, preferably less than about 20 μm—and can include any suitable substrate polymer that is compatible with thin film micromachining, provides good electrical insulation and encapsulation for electrical leads in wet ionic environments, is biocompatible, is sufficiently strong to allow handling, and has a sufficiently low Young's modulus to allow strong bending during hydrogel expansion/contraction. In a preferred embodiment, the flexible polymer is polyimide. The polymer can additionally, or alternatively, include parylene C or other biocompatible polymers.

In some embodiments, a sufficiently low Young's modulus is determined as being high enough to resist bending due to the shear force of fluid flow in an in vivo environment (e.g., to resist bending from the shear force of blood flow) and low enough that the hydrogel can bend the polymer. Accordingly, the length and thickness of the hydrogel can affect the requisite properties of the flexible polymer substrate, including for example, the Young's modulus of the flexible polymer substrate. In the same respect, the thickness of the hydrogel can affect the response time of the hydrogel to analyte concentrations, and the surface to volume ratio of the hydrogel can be balanced with properties of the flexible polymer substrate based on the desired implementation.

In some embodiments, the stimulus-responsive hydrogel is deposited on a distal end of the sensing structure as one or more micro-strips. The geometry of each hydrogel micro-strip can vary. In some embodiments, one or more of the micro-strips is preferably between about 2 μm-200 μm thick, about 25 μm-500 μm wide, and about 500 μm-1000 μm long. For example, a hydrogel micro-strip can be about 20 μm thick, about 100 μm wide, and about 500 μm long. As an additional example, a hydrogel micro-strip can be less than about 100 μm thick, between about 50 μm-200 μm wide, and at least about 500 μm long.

Polyimide can be used in catheter applications that involve thin walls and high mechanical strength and is a well-adapted polymer thin film technology in microfabrication with good adhesion to thin film metals. In some embodiments, Kapton® polyimide is used and provides high temperature processing (e.g., during steam sterilization: 121/135° C., 2 bar) without permanent negative impact on electrical and mechanical properties. In some embodiments, the thin film metal is made of or includes gold, platinum, titanium, aluminum, and alloys thereof. In one embodiment, the metal is a platinum-titanium alloy. A Pt/Ti thin film metallization exhibits excellent biocompatibility, corrosion resistance, and adhesion to polyimide while providing acceptable electrical conductivity.

In some embodiments, the sensing structure includes a three-dimensional stack of a plurality of polymer-metal structures. Each layer of the three-dimensional stack can be isolated between intervening polymer layers and/or interconnected at various positions within the stack.

Additionally, the sensing structure can include any number and/or length of conductive meandering leads and can be associated with any number or positioning of capacitors.

### Methods for Manufacturing Thin-Film-Based Sensor Devices

Embodiments of the present disclosure additionally include methods for manufacturing thin-film-based sensor devices, such as those described above with respect to FIG. 5. For example, an exemplary manufacturing paradigm for thin-film-based sensor devices is illustrated by the flow diagram of FIG. 7. As shown, a method 700 for manufacturing a sensor device includes a plurality of method acts (acts 702-712). The method 700 can include depositing a first polymer layer of a flexible thin film polymer onto a carrier wafer (act 702). The carrier wafer can be made of any suitable material, preferably silicon, and the polymer can be any suitable polymer described herein, preferably polyimide.

The method 700 can further include selectively depositing a metal thin film pattern on a portion of the first polymer layer (act 704). The metal thin film can include any suitable metal described herein and can be deposited in any pattern to form an electric sensing element capable of electric impedance change. For example, the metal thin film can be deposited as a single conductive meandering lead or a plurality of conductive meandering leads.

Method 700 additionally includes depositing a second polymer layer over the first polymer layer and over the metal thin film (act 706). After depositing the second polymer layer as described in act 706, a polymer-metal structure is formed that contains the metal thin film and the flexible thin film polymer, though still attached to the carrier wafer. The remaining components of the sensing structure are formed in method act 708 by fabricating contact pads and creating an outline of the sensing structure to include the contact pads. This process can include dry etching the outline and one side of the polymer layer to create bond pads, the metal itself acting as an etch stop layer of the sensing structure and removing it from the carrier wafer.

The method 700 of manufacturing a sensor device can additionally include chemically treating a firsts side of the sensing structure (act 710). The sensing structure can be chemically treated with any suitable chemical that increases adhesion of the hydrogel to the surface, as method 700 subsequently includes molding and conditioning a stimulus-responsive hydrogel to the sensing structure (act 712), preferably on the first, chemically treated side thereof. For example, the hydrogel can be micro-dispensed in a desired thickness (e.g., 20 μm-50 μm) and in situ light cured or thermally polymerized on the surface of the sensing structure to form the sensor device.

### Exemplary Applications of Disclosed Sensor Devices

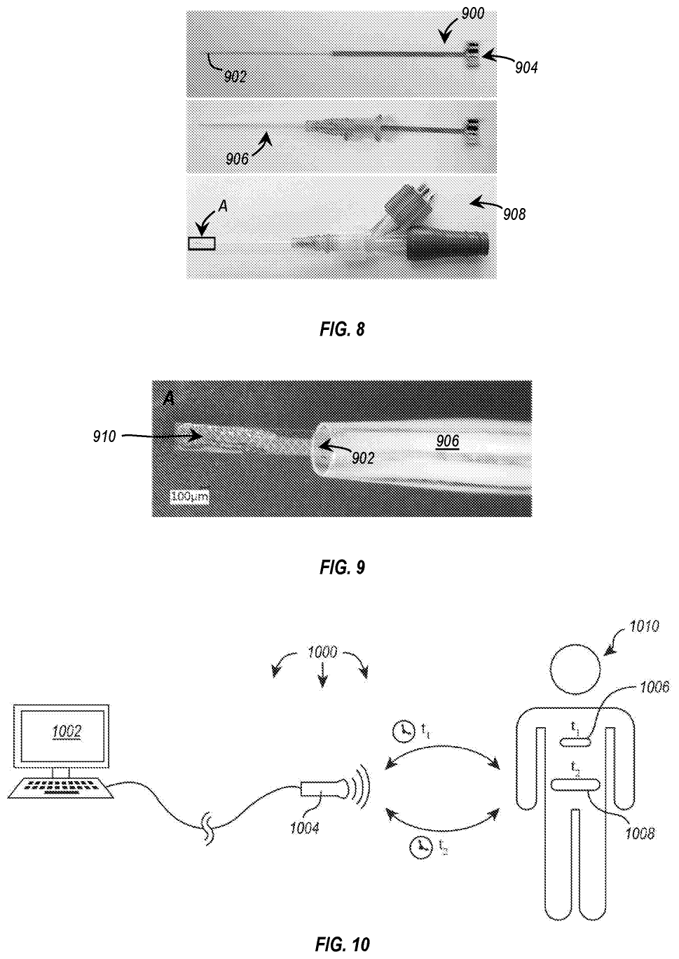

Embodiments of the present disclosure can be implemented in vivo to measure the values/concentrations of various blood/plasma analytes, which can be particularly beneficial during surgery for real-time measurement of analyte concentration. In one embodiment, the sensor devices disclosed herein can be incorporated as part of a catheter (or similar item) to collect data in the blood stream with little to no negative impact on patient risk and comfort, care provider workflow or complexity, and at a manufacturing cost comparable to non-sensing catheters. As shown in FIGS. 8 and 9, a sensor device 900 can include a sensing portion with a stimulus-responsive hydrogel 902 associated therewith and a set of contact pads 904 for electrically coupling the sensor device to a circuit (for wireless transmission) or to a meter (for measuring and monitoring electrical properties of the sensor device). The sensor device 900 can be placed in the center lumen of a catheter sheath 906, where it occupies less than about 10% of the inner lumen, preferably less than 5% of the inner lumen, or more preferably less than about 2.5% of the inner lumen. It should be appreciated that the substrate width can be determined by the inner diameter of the hollow injection needle initially inside the sheath but retracts following insertion of the sheath.

Once placed in the lumen, the sensor tip can protrude beyond the opening of the catheter sheath, as shown in the close-up view of the distal end of the catheter assembly 908 in FIG. 9. In some embodiments, the sensor tip extends about 2 mm beyond the opening of the catheter sheath for placement in the center of the blood stream with limited risk of touching blood vessel walls where blood flow is reduced and exchange of analytes with the hydrogel could be hindered. The thin polymer material of the sensor device is additionally configured to exert a negligible force on tissue if brought into contact, as compared to the catheter sheath.

In some in vivo implementations, the hydrogel may be prone to breaking off—in parts or completely—from the thin polymer sensing structure, leading to the release of small particles that may potentially increase the risk of secondary events (e.g., stroke). Accordingly, in some embodiments, commercial dialysis catheter membranes can be placed as a cap over the tip preventing the release of pieces into the blood stream without limiting fluid flow and analyte exchange between blood and hydrogel.

In one embodiment, the sensor device can be included as part of a temporary or chronic intra-venous catheter. Such embodiments can be particularly useful, for example, in measuring biomarkers in the blood of patients under general anesthesia during surgery or who are in stationary or ambulant care. For example, the sensor device can be used to measure pH, osmolality, glucose concentration, sodium concentration, potassium concentration, lactate concentration, hemoglobin levels, calcium concentration, bicarbonate levels, pO₂ levels, or pCO₂ levels in the blood of a patient with a temporary or chronic catheter. Additionally, or alternatively, the sensor can detect one or more proteins of the coagulation cascade, such as thrombin and Factor Xa. Combinations with neurological markers can additionally be used to more accurately determine anesthesia level and reduce patient risk.

In some embodiments, multiple sensors are used at the same time to measure a plurality of analytes/biomarkers. The sensor can be multiplexed or added serially and owing to the limited space occupied by individual sensors, it is possible to have multiple sensor devices associated with a single catheter sheath without significantly compromising the utility of the catheter to deliver therapeutics into the bloodstream or to draw blood therefrom. These and other disclosed embodiments advantageously provide more pharmacokinetic, real-time, and quantitative data that can be used to treat patients more appropriately and accurately but also to intervene for rescue. As a specific, non-limiting example, the stimulus-responsive hydrogel associated with the sensor device can be specific for one or more active components of general anesthesia or metabolic derivatives thereof, allowing an anesthesiologist or other healthcare provider to more accurately monitor the amount or concentration of anesthesia in the patient's bloodstream and to adjust one or more drugs according to the patient's individual ability to metabolize and/or filter the drugs from their system. A similar approach can be adapted for use in patients prescribed restricted drugs or drugs associated with a high incidence of abuse and/or addiction (e.g., opioids, amphetamines, etc.). The amount or concentration of the prescribed medication can be monitored to ensure compliance and/or to prevent abuse.

In some embodiments, current measurement methods and sensor devices fail to provide sufficient advance notice of a potentially threatening event or worsening condition. For example, opioids cause hypopnea, hypercapnia, and respiratory acidosis with normal oxygen saturation by decreasing brainstem responsiveness to carbon dioxide. As a result, pulse oximetry lags in predicting hypoventilation from respiratory depression. Increasing pCO₂ levels during sedation can be a better predictor of hypoventilation, and thus life-threatening hypoxemia, yet quickly and accurately measuring pCO₂ has heretofore been problematic.

End-tidal CO₂ monitoring allows for an objective measurement of expired CO₂ but exhaled levels can be difficult to detect or monitor in awake or non-intubated patients. Arterial blood gas measurements, as a gold standard, are invasive, discontinuous, require laboratory analysis, and are thus hard to synchronize. Further, they can suffer from sampling errors due to heparin use and exposure to air. Transcutaneous carbon dioxide measurements use heated electrochemical sensors applied to the skin. They depend on tissue perfusion at the probe site and thus can lead to inaccurate measurements when hemodynamic status is compromised. At least some of these problems, among others in the art, are addressed by the disclosed sensor devices and systems, which provide reliable, real-time blood CO₂ monitoring for the detection of hypercapnia and possible opioid-induced apnea or hypopnea.

For example, sensor devices of the present invention can be configured to measure pCO₂ levels in addition to the level of opiate concentration in the patient's blood. In some embodiments, the sensor devices can measure the combination of ingested opioids or sedatives individually or collectively to provide a treating physician with a more robust way of maintaining opioid levels in the blood and/or treating the patient for opioid abuse. In some embodiments, the sensor device is sensitive to fentanyl and/or pCO₂ levels. By measuring pCO₂ levels, over dosage of opioid use that leads to suppression of breathing can be detected prior to respiratory arrest and can quickly and reliably measure compliance and opioid effect in patients.

Referring now to FIG. 10, illustrated is a system 800 for identifying one or more changes over time in a stimulus-responsive hydrogel associated with a sensor device 1006, 1008 positioned within an in vivo environment (e.g., inside the human body). The system 800 includes a sensor device 1006, 1008 positioned within an in vivo environment 1010, a meter for determining one or more electric properties of the sensor device 1006, 1008, and a computing system 1002 in electrical communication with the meter 1004. At a first time (ti), the meter 1004 identifies an electrical property of the sensor device 1006 within the in vivo environment 1010. The computing system 1002 can identify and/or record one or more characteristics of the sensor device 1006 at the first time (ti).

In some implementations, and for the purposes of FIG. 10, the stimulus-responsive hydrogel associated with the sensor device 1006 can reach a physiologic equilibrium with one or more stimuli (e.g., pH, osmolarity, temperature, etc.) and the levels (or non-existence) of analyte within the in vivo system 1010 prior to identifying and/or recording one or more characteristics of the sensor device 1006 at the first time (t₁). At a second time (t₂)—the second time coming chronologically after the first time (t₁)—the meter 1004 determines one or more electric properties of the sensor device 1008 in the in vivo environment 1010, and the computing system 1002 identifies and/or records one or more updated characteristics of the sensor device 1008. As shown in FIG. 10, the sensor device 1008 has changed physical properties compared to the sensor device at the first time 1006, which is indicative of the sensor device bending or stretching in response to the associated stimulus-responsive hydrogel expanding or contracting.

In some embodiments, the meter 1004 communicates with the sensor device wirelessly, as shown in FIG. 10. Alternatively, the sensor device can be directly coupled to the meter.

Although the in vivo environment 1010 is generically depicted in FIG. 10 as being within the human body, it should be appreciated that the in vivo environment can include any number or types of locations within the human body. For example, the sensor device can be placed within the bloodstream to identify an amount or concentration of any of the analytes described above, including the presence or concentration of opioids or opioid byproducts in the blood.

Other implementations within the blood include monitoring a stimulus-responsive hydrogel that is specific for peptidoglycan, mycolic acid, viral capsid proteins, or other bacterial and/or viral specific antigens as a means of noninvasively monitoring the patient for bacteremia, viremia, or other blood-based infection. This can increase the response time of attending medical professionals to potentially life-threatening conditions, particularly in immunocompromised patients, so that the infection can be caught and treated early on where the prognosis for recovery is best. This can be particularly impactful, for example, in patients at risk for septicemia, where survival rates are inversely and exponentially correlated with the number of hours the patient is septicemic.

It should be appreciated that while the foregoing disclosure has focused mainly on humans, the same or analogous stimulus-responsive hydrogels can be used within in vivo environments of nonhuman animals.

Additionally, or alternatively, implementations can include the use of stimulus-responsive hydrogels within environmental locations. This may be particularly advantageous when monitoring, for example, water lines for the presence of pollutants (e.g., toxic metals, known carcinogens, etc.), microbial contamination, or additives (e.g., fluoride). Multiple stimulus-responsive hydrogels can be placed at critical points within a supply line and quickly and noninvasively monitored. In some implementations, strategic placement of stimulus-responsive hydrogels within environmental locations (e.g., along a water way or within a water line) can assist in and/or expedite identification of a contaminating source or other issue.

In some implementations, the sensor device can include a stimulus-responsive hydrogel that is multifaceted such that the hydrogel swells in response to interaction with any one of the plurality of analytes. This may be advantageous, for example, in situations where the sensor device is used identify pollutants or contaminants, any of which alone may be cause for concern and/or the confluence of all should be below the prescribed threshold. In this way, a single sensor device can be placed and monitored (locally and/or remotely) as opposed to deploying a plurality of individually specific sensor devices to accomplish the same or similar purpose. As such, the foregoing implementations can save time and provide a more effective use of resources.

It should be appreciated that many of the embodiments of the present invention can include or utilize a special purpose or general-purpose computer, including computer hardware, to perform various method acts disclosed herein (e.g., determining the concentration of an analyte, monitoring an analyte concentration, etc.). Accordingly, embodiments within the scope of the present invention may include physical and other computer-readable media for carrying or storing computer-executable instructions and/or data structures. Such computer-readable media can be any available media that can be accessed by a general purpose or special purpose computer system. Computer-readable media that store computer-executable instructions are physical storage media. Computer-readable media that carry computer-executable instructions are transmission media. Thus, by way of example, and not limitation, embodiments of the invention can comprise at least two distinctly different kinds of computer-readable media: physical computer-readable storage media and transmission computer-readable media.

Physical computer-readable storage media includes RAM, ROM, EEPROM, CD-ROM or other optical disk storage (such as CDs, DVDs, etc.), magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry or desired program code means in the form of computer-executable instructions or data structures and which can be accessed by a general purpose or special purpose computer. Combinations of the above are also included within the scope of computer-readable media.

Further, upon reaching various computer system components, program code means in the form of computer-executable instructions or data structures can be transferred automatically from transmission computer-readable media to physical computer-readable storage media (or vice versa). For example, computer-executable instructions or data structures received over a network or data link can be buffered in RAM within a network interface module (e.g., a “NIC”), and then eventually transferred to computer system RAM and/or to less volatile computer-readable physical storage media at a computer system. Thus, computer-readable physical storage media can be included in computer system components that also (or even primarily) utilize transmission media.

Computer-executable instructions comprise, for example, instructions and data which cause a general purpose computer, special purpose computer, or special purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, and the like. The invention may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Alternatively, or in addition, the functionality described herein can be performed, at least in part, by one or more hardware logic components. For example, and without limitation, illustrative types of hardware logic components that can be used include Field-programmable Gate Arrays (FPGAs), Program-specific Integrated Circuits to (ASICs), Program-specific Standard Products (AS SPs), System-on-a-chip systems (SOCs), Complex Programmable Logic Devices (CPLDs), etc.

### Conclusion

Any headings used herein are for organizational purposes only and are not meant to be used to limit the scope of the description or the claims.

Various aspects of the present disclosure, including devices, systems, and methods may be illustrated with reference to one or more embodiments or implementations, which are exemplary in nature. As used herein, the term “exemplary” means “serving as an example, instance, or illustration,” and should not necessarily be construed as preferred or advantageous over other embodiments disclosed herein. In addition, reference to an “implementation” of the present disclosure or invention includes a specific reference to one or more embodiments thereof, and vice versa, and is intended to provide illustrative examples without limiting the scope of the invention, which is indicated by the appended claims rather than by the following description.

As used throughout this application the words “can” and “may” are used in a permissive sense (i.e., meaning having the potential to), rather than the mandatory sense (i.e., meaning must). Additionally, the terms “including,” “having,” “involving,” “containing,” “characterized by,” as well as variants thereof (e.g., “includes,” “has,” “involves,” “contains,” etc.), and similar terms as used herein, including within the claims, shall be inclusive and/or open-ended, shall have the same meaning as the word “comprising” and variants thereof (e.g., “comprise” and “comprises”), and do not exclude additional un-recited elements or method steps, illustratively. Unless defined otherwise, all technical and scientific terms used herein have the same meaning as commonly understood by one of ordinary skill in the art to which the present disclosure pertains.

Disclosure of certain features relative to a specific embodiment of the present disclosure should not be construed as limiting application or inclusion of said features to the specific embodiment. Rather, it will be appreciated that other embodiments can also include said features, members, elements, parts, and/or portions without necessarily departing from the scope of the present disclosure. Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

Accordingly, the present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatus disclosed herein may be made without departing from the scope of the disclosure or of the invention, which is defined in the appended claims. All changes which come within the meaning and range of equivalency of the claims are to be embraced within their scope.

What is claimed is:

1. A sensor device for identifying one or more changes in a stimulus-responsive hydrogel, the sensor device comprising:
a sensing structure comprising a flexible thin film polymer and an electric sensing element capable of electric impedance change; and
a stimulus-responsive hydrogel associated with a first side of the sensing structure, the stimulus-responsive hydrogel configured to dimensionally change in response to one or more predefined stimuli, wherein a dimensional change of the stimulus-responsive hydrogel causes a change in one or more impedance properties of the electric sensing element.

2. The sensor device of claim 1, wherein the electric sensing element comprises a conductive meandering lead embedded within the flexible thin film polymer substrate.

3. The sensor device of claim 2, wherein the conductive meandering lead comprises a metal thin film, the metal being selected from the group consisting of gold, platinum, titanium, aluminum, and alloys thereof.

4. The sensor device of claim 1, wherein the electric sensing element comprises a three-dimensional stack of interconnected conductive meandering leads embedded within the flexible thin film polymer.

5. The sensor device of claim 1, wherein the stimulus-responsive hydrogel is deposited on a distal end of the sensing structure such that the dimensional change of the stimulus-responsive hydrogel causes the change in the one or more impedance properties of the electric sensing element by bending the sensor substrate.

6. The sensor device of claim 5, wherein the stimulus-responsive hydrogel is deposited on the distal end of the sensing structure as one or more micro-strips, each of the one or more micro-strips being between about 2 μm-200 μm thick, about 25 μm-500 μm wide, and about 500 μm-1000 μm long.

7. The sensor device of claim 6, wherein the flexible thin film polymer substrate comprises a thin film polyimide substrate between about 10 μm-20 μm thick.

8. A system for measuring one or more changes in a stimulus-responsive hydrogel positioned within an in vivo environment, the system comprising the sensor device of claim 5 and a catheter sheath configured for placement within the in vivo environment, the catheter sheath being sized and shaped to receive the sensor device within a lumen of the catheter sheath.

9. A system for measuring one or more changes in a stimulus-responsive hydrogel positioned within an in vivo environment, the system comprising the sensor device of claim 5 and a wireless receiver configured to communicate with the sensor device positioned in an in vivo environment.

10. A method for manufacturing the sensor device of claim 5, comprising:
depositing a first polymer layer of the flexible thin film polymer on a carrier wafer;
selectively depositing a metal thin film pattern comprising the electric sensing element on a portion of the first polymer layer;
depositing a second polymer layer of the flexible thin film polymer over the first polymer layer and the metal thin film pattern;
fabricating one or more contact pads and creating an outline of the sensing structure, wherein the sensing structure additionally comprises the one or more contact pads;
chemically treating the first side of the sensing structure; and
molding and conditioning the stimulus-responsive hydrogel on the first side of the sensing structure.

11. A system for measuring one or more changes in a stimulus-responsive hydrogel positioned within an in vivo environment, the system comprising:
a sensing structure comprising an electric sensing element capable of electric impedance change;
a stimulus-responsive hydrogel associated with the sensing structure, the stimulus-responsive hydrogel configured to dimensionally change in response to one or more predefined stimuli; and
a meter in electrical communication with the sensing structure, the meter configured to identify a change in one or more impedance properties of the electric sensing element.

12. The system of claim 11, wherein the electric sensing element comprises a micro-coil.

13. The system of claim 12, wherein the sensing electric sensing element comprises a resonance circuit having a capacitor connected to the micro-coil.

14. The system of claim 12, wherein the micro-coil comprises a soft metal wire having a diameter between about 5 μm-50 μm.

15. The system of claim 12, wherein the stimulus-responsive hydrogel surrounds the micro-coil, and wherein a dimensional change within the stimulus-responsive hydrogel causes a compression or lengthening of the micro-coil.

16. The system of claim 12, wherein the stimulus-responsive hydrogel comprises a thin strand hydrogel core disposed within an internal space defined by the micro-coil, and wherein a dimensional change within the stimulus-responsive hydrogel causes a change in a magnetic permeability of the micro-coil.

17. The system of claim 16, wherein the thin strand hydrogel core comprises a plurality of magnetic particles.

18. The system of claim 11, further comprising a computer system in electrical communication with the meter, the computer system having one or more processors and being configured to:
receive, from the meter, a first inductance of the sensing structure at a first point in time and a second inductance of the sensing structure at a second point in time; and
calculate, at the one or more processors, a volumetric change in the stimulus-responsive hydrogel based on the received first and second inductances.

19. A method for measuring a concentration of an analyte within an in vivo environment, the method comprising:
positioning a sensor device within the in vivo environment, the sensor device comprising a sensing structure and a stimulus-responsive hydrogel associated with the sensing structure, the stimulus-responsive hydrogel configured to change one or more impedance properties of the sensing structure in response to the concentration of the analyte;
receiving, from a meter in electrical communication with the sensor device, a first impedance of the sensing structure;
measuring an impedance amplitude or phase change; and
determining the concentration of the analyte based on the impedance amplitude or phase change.

20. The method of claim 19, further comprising:
receiving, from the meter in electrical communication with the sensor device, a second impedance of the sensing structure;
calculating an impedance change based on the first impedance and second impedance; and
determining an updated concentration of the analyte based on the impedance change.
